(19) United States
(12) Patent Application Publication
(10) Pub. No.: US 2026/0241168 A1
(43) Pub. Date: Aug. 20, 2026

(54) MULTI-SITE NEUROMODULATION

(71) Applicant: Medtronic, Inc., Minneapolis, MN (US)

(72) Inventor: David J. Miller, Austin, TX (US)

(21) Appl. No.: 19/161,468

(22) PCT Filed: Mar. 1, 2024
(86) PCT No.: PCT/IB2024/052006

§ 371 Date: Sep. 2, 2025

Related U.S. Application Data
(60) Provisional application No. 63/489,356, filed on Mar. 9, 2023.

(51) Int. Cl. A61N 1/36; A61N 1/02; A61N 1/04; A61N 1/05

(52) U.S. Cl. CPC A61N 1/36031; A61N 1/025; A61N 1/0456; A61N 1/0492; A61N 1/0517; A61N 1/36017

(57) ABSTRACT

Example systems, devices and techniques are disclosed for delivering neurostimulation therapy to multiple sites. An example system or device includes stimulation circuitry configured to generate a first stimulation signal and a second stimulation signal and memory configured to store first stimulation parameters that at least partially define the first stimulation signal and second stimulation parameters that at least partially defined the second stimulation signal. The example system or device also includes processing circuitry communicatively coupled to the memory, and the stimulation circuitry. The processing circuitry is configured to control the stimulation circuitry to generate the first stimulation signal configured to be delivered transcutaneously or percutaneously to a first anatomical location and control the stimulation circuitry to generate the second stimulation signal configured to be delivered transesophageally to a second anatomical location different than the first anatomical location.

This Application claims priority from U.S. Provisional Patent Application 63/489,356, filed 9 Mar. 2023, the entire content of which is incorporated herein by reference.

### TECHNICAL FIELD

The disclosure relates to devices and techniques for stimulating a plurality of nerves.

### BACKGROUND

A stroke may be defined as damage to the brain from the interruption of the blood supply of the brain. This damage results directly from a lack of blood perfusion, but also from the ensuing inflammation from the ischemia or ischemia-reperfusion injury.

Neuromodulation by electrical stimulation of the cervical, thoracic, and abdominal branches of the vagus nerve has been shown to be useful for a wide range of purposes. Neuromodulation of the trigeminal nerve has been shown to increase cerebral perfusion.

### SUMMARY

Recent research has revealed that trigeminal nerve stimulation (TNS) has the potential to increase cerebral perfusion. The mechanism of action is hypothesized to be related to the dive reflex present in mammals which may result in reduced cerebral vascular resistance, which in turn, increases cerebral blood flow. The dive reflex is a physiological reflex that occurs when, for example, one holds their breath and submerges under water.

Increasing cerebral blood flow may augment collateral circulation to the zone near the infarct (e.g., the region of the brain with no blood circulation) called the penumbra. The penumbra may be a zone of brain tissue around the ischemic core that may be subject to potential damage (e.g., including reversable damage) and may be considered an at-risk zone. By enhancing the penumbra perfusion, a greater portion of the penumbra may survive.

Separately, vagus nerve stimulation (VNS) has been shown to trigger an anti-inflammatory reflex that reduces damage from an ischemia-reperfusion injury, such as a stroke. The benefit of VNS is primarily from activation of the cholinergic anti-inflammatory pathway, though other pathways and mechanisms of action may exist. VNS does not appear to alter hemodynamics. For example, stimulation of the abdominal vagus does not generally change the average blood pressure, heart rate, or respiration rate.

In general, this disclosure is directed to devices, systems, and techniques for stimulating a plurality of sites to treat a malady of a patient, such as stroke. An example system may treat the patient by stimulating a vagus nerve and stimulating a trigeminal nerve of the patient at different locations. Because the vagus nerve is located in an anatomically challenging location to access, a vagus nerve may be challenging to stimulate without an invasive surgical procedure, which may be undesirable for situations including an acute illness, a short duration of stimulation, or when reduced time to stimulation is important to patient treatment. As such, the system may stimulate the vagus nerve in a less invasive manner, such as transesophageally, via one or more electrodes disposed within the esophagus of the patient. The system may stimulate the trigeminal nerve transcutaneously and/or percutaneously. The system of this disclosure may be used to treat stroke, traumatic brain injury (TBI), subarachnoid hemorrhage (SAH), or the like.

In one example, the disclosure is directed to a system including: stimulation circuitry configured to generate a first stimulation signal and a second stimulation signal; memory configured to store first stimulation parameters that at least partially define the first stimulation signal and second stimulation parameters that at least partially defined the second stimulation signal; and processing circuitry communicatively coupled to the memory, and the stimulation circuitry, the processing circuitry being configured to: control the stimulation circuitry to generate the first stimulation signal configured to be delivered transcutaneously or percutaneously to a first anatomical location; and control the stimulation circuitry to generate the second stimulation signal configured to be delivered transesophageally to a second anatomical location different than the first anatomical location.

In another example, this disclosure is directed to a system including stimulation circuitry configured to generate a first stimulation signal and a second stimulation signal; memory configured to store first stimulation parameters that at least partially define the first stimulation signal and second stimulation parameters that at least partially defined the second stimulation signal; first electrodes configured to transcutaneously or percutaneously deliver the first stimulation signal to a first nerve of a patient; second electrodes configured to transesophageally deliver the second stimulation signal to a second nerve of the patient; and processing circuitry communicatively coupled to the memory, and the stimulation circuitry, the processing circuitry being configured to: control the stimulation circuitry to generate the first stimulation signal; and control the stimulation circuitry to generate the second stimulation signal.

In another example, this disclosure is directed to a non-transitory computer readable medium comprising instructions, which when executed, cause processing circuitry to control stimulation circuitry to generate a first stimulation signal, the first stimulation signal being at least partially defined by first stimulation parameters and being configured to be delivered transcutanously or percutaneously to a first anatomical location; and control the stimulation circuitry to generate a second stimulation signal, the second stimulation signal being at least partially defined by second stimulation parameters and being configured to be delivered transesophageally to a second anatomical location different than the first anatomical location.

The details of one or more examples are set forth in the accompanying drawings and the description below. Other features, objects, and advantages of the disclosure will be apparent from the description and drawings, and from the claims.

The above summary is not intended to describe each illustrated example or every implementation of the present disclosure.

### BRIEF DESCRIPTION OF DRAWINGS

FIG. 1 is a conceptual diagram illustrating an example trigeminal nerve of a patient in accordance with one or more aspects of this disclosure.

FIG. 2 is a conceptual diagram illustrating an example vagus nerve of a patient in accordance with one or more aspects of this disclosure.

FIG. 3 is a conceptual diagram illustrating an example multi-site neurostimulation system 10 according to the techniques of this disclosure.

FIG. 4 is a conceptual diagram illustrating an example cross section of elongated member according to the techniques of this disclosure.

FIG. 5 is a block diagram of an example controller for a multi-site neurostimulation system according to one or more aspects of this disclosure.

FIG. 6 is a block diagram illustrating an example configuration of a computing device in accordance with one or more aspects of this disclosure.

FIG. 7 is a flow diagram illustrating example multi-site neurostimulation techniques in accordance with one or more aspects of this disclosure.

### DETAILED DESCRIPTION

The present disclosure is directed to devices, systems, and techniques for multi-site neurostimulation. The devices, systems, and techniques of this disclosure may deliver both TNS and VNS to a patient, for treatment of a patient malady, such as a stroke, TBI, or SAH. The TNS may be configured to increase blood flow to the brain, while the VNS may be configured to decrease inflammation. Thus, the multi-site neurostimulation may improve patient outcomes by preserving the penumbra.

The anatomical location of the vagus nerve makes the vagus nerve difficult to stimulate without an invasive surgical procedure. In an acute situation, such as during other surgery or an abrupt illness or injury, such as stroke, TBI, SAH, or when the required duration of stimulation is limited to relatively short amount of time, it may be undesirable to undertake an invasive surgical procedure to implant a stimulation device. Recent discoveries relating to VNS have uncovered the nervous system involvement and control of the body's inflammatory response. The nervous system senses inflammation, pathogens, and tissue damage, as well as modulates the response to such sensed issues. One pathway of the nervous system is referred to as the cholinergic anti-inflammatory pathway (CAP). Stimulating certain nerves, usually branches of the vagus nerve, can dampen the inflammatory response and associated cytokines. Stimulation in the cervical vagus, the abdominal vagus, the auricular branch of the vagus in the ear, the sacral nerve, the tibial nerve, and others can be used in some scenarios. Varying the stimulation, inflammatory cytokines can be modulated up or down.

Implantable cervical vagus stimulators are commercially available for the treatment of epilepsy, but involve complex and invasive surgery to implant the stimulating electrode on the nerve. Other technologies attempt to stimulate the vagus transcutaneously with an external device, but those may have limited success due to the distance from the skin surface to the vagus nerve, the potency of stimulation, and/or the usability of holding a device in the proper position.

As such, it may be desirable to have a system and techniques for stimulating the cervical, thoracic, or abdominal vagus nerve branches and the trigeminal nerve that is easy and quick to use. Such a system and techniques would be useful for short-term stimulation, such as in response to stroke, TBI, SAH, or the like.

A device configured to stimulate the trigeminal nerve and the vagus nerve without an invasive surgical procedure may be used to treat a variety of illnesses, including but not limited to: stroke, TBI, SAH, or the like. In some examples, such a device may also be used to treat surgical or non-surgical acute kidney injury, postoperative ileus, postoperative cognitive decline or postoperative delirium, asthma, sepsis, bleeding, myocardial infarction, dysmotility and obesity, or the like. For example, TNS may increase cerebral blood flow. VNS may trigger the cholinergic anti-inflammatory pathway (CAP). CAP has been shown to reduce excessive inflammation and would be useful for treating a variety of illness including, treating any of these diseases or conditions may improve patient outcomes, shorten length of hospital stays, and/or reduce medical costs.

Many conditions can be caused by damage to tissue from an overreaction of the inflammatory process. One such condition is ischemia-reperfusion injury (IRI). In IRI, tissue experiences ischemia due to reduced or stopped blood supply, followed by reperfusion due to medical intervention (e.g., when a blood clot causing a stroke is removed) or the body's healing response. When tissue experiences an IRI, the immune system reacts to the damaged or dead cells with an intense inflammatory response causing infarcted tissue and loss of long-term function. Reducing the inflammatory response during the ischemia or reperfusion can reduce the resulting infarct volume and improve function. Examples of common conditions that may lead to IRI include acute ischemic stroke and transient ischemic attack (the blockage of blood vessels in the brain or leading to the brain), myocardial infarction (the abrupt blockage of coronary arteries leading to zones of infarcted heart tissue), or acute kidney injury (AKI) (abrupt loss of renal function due to an injury). AKI typically occurs in surgical patients and septic patients. AKI is distinct from chronic kidney disease, which is the gradual loss of kidney function. AKI can be caused by many things, but a common cause is reduced renal blood flow and/or renal blood oxygen extraction.

There are other common acute medical problems that involve an inappropriate overreaction by the immune system. Some examples of such conditions include severe asthma attacks with may involve excessive mucus secretion and airway narrowing, sepsis (some forms of sepsis may be driven by the immune system over-reacting, which may be referred to as a “cytokine storm”), or post-operative ileus. For example, after abdominal surgeries, it is common for patients to have ileus, or the inability of the intestine (bowel) to contract normally and move waste out of the body. Ileus may be caused by an inflammatory response in the bowel due to surgical manipulation.

Other conditions manifest as an imbalance in the sympathetic/parasympathetic balance. If the imbalance is decreased parasympathetic tone, the imbalance can cause temporary cardiac arrhythmias.

All of the above conditions may be treated (e.g., reduced symptoms or improved clinical outcomes) by stimulating the vagus nerve. VNS may reduce inflammatory damage from IRI, return inflammation to a normal level and prevent the hyperinflammatory response, and/or restore a healthy, normal parasympathetic/sympathetic balance.

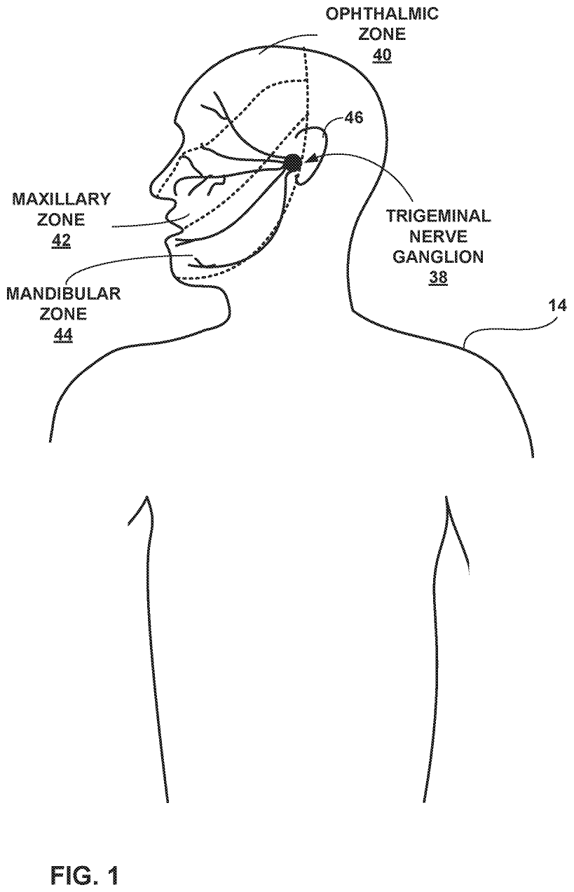

FIG. 1 is a conceptual diagram illustrating an example trigeminal nerve of a patient in accordance with one or more aspects of this disclosure. Patient 14 is depicted with ear 46 for anatomical reference. Trigeminal nerve ganglion 38 of patient 14 may be located near or under ear 46. The trigeminal nerve is shown with various branches traversing the head/facial area of patient 14 from trigeminal nerve ganglion 38. The area in which the branches of the trigeminal nerve 38 be located may be divided into three regions or zones: ophthalmic zone 40 (e.g., the V1 branch), maxillary zone 42 (e.g., the V2 branch), and mandibular zone 44 (e.g., the V3 branch). These zones are shown divided by the dotted lines in FIG. 1.

TNS devices have been developed for the treatment of migraines and other disorders. TNS stimulation may be provided by non-invasive electrodes applied to the skin (e.g., via one or more patches) or with penetrating needle electrodes. In some examples, a patch may include an adhesive for securing the patch to the outer surface of the skin.

The application of neurostimulation to specific locations on the trigeminal nerve have been shown to have different stimulation efficacy. Research has shown that neurostimulation to ophthalmic zone 40 may have a larger effect on cerebral blood flow than neurostimulation to maxillary zone 42, mandibular zone 44, or trigeminal nerve ganglion 38. However, neurostimulation to any of these zones/ganglions is contemplated herein.

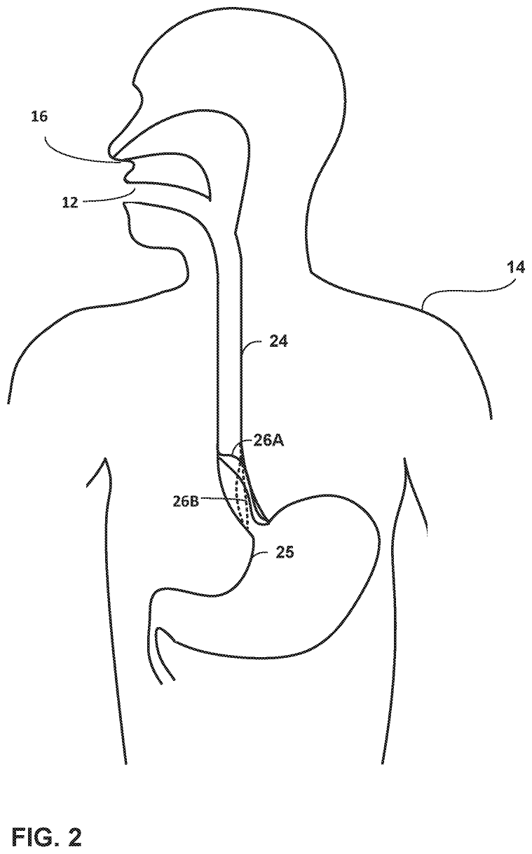

FIG. 2 is a conceptual diagram illustrating an example vagus nerve of a patient in accordance with one or more aspects of this disclosure. Patient 14 is depicted having stomach 25 and esophagus 24. Mouth 12 and nasal cavity 16 are connected to esophagus 24 and may provide access to esophagus 24 for a transesophageal neurostimulation system (not shown). Also depicted are representations of branches of the vagus nerve, namely anterior branch 26A of the vagus nerve or posterior branch 26B of the vagus nerve. A device may deliver neurostimulation transesophageally to one or more of anterior branch 26A of the vagus nerve or posterior branch 26B of the vagus nerve via one or more electrodes disposed within esophagus 24 of patient 14.

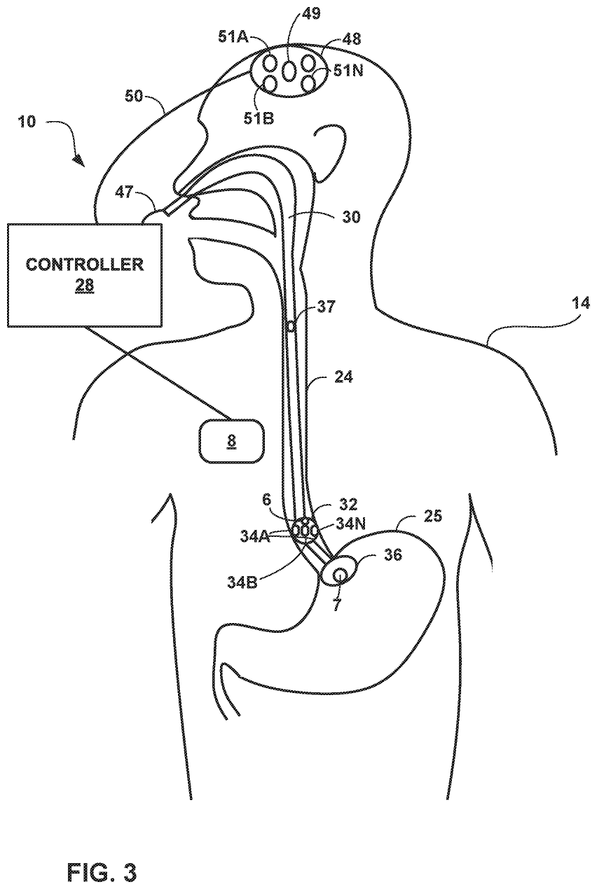

FIG. 3 is a conceptual diagram illustrating an example multi-site neurostimulation system 10 according to the techniques of this disclosure. Multi-site neurostimulation system 10 may include a controller 28 for controlling neurostimulation. Multi-site neurostimulation system 10 may deliver TNS to a location of the trigeminal nerve (e.g., ophthalmic zone 40 of FIG. 1) and may deliver VNS to a location of the vagus nerve (e.g., anterior branch 26A and/or posterior branch 26B) transesophageally. While this disclosure primarily discusses the delivery of VNS transesophageally, in some examples, multi-site neurostimulation system 10 may deliver VNS in another manner, such as transcutaneously. In some examples, multi-site neurostimulation system 10 may be implemented and sold as a single device capable of independently, but simultaneously providing VNS and TNS where the VNS is transesophageal and the TNS is transcutaneous. In some examples, multi-site neurostimulation system 10 may be delivered to deliver VNS and TNS at different, non-overlapping times, or at different times that may at least partially overlap in time. Such a device may include a nasogastric tube. The device may include a single nerve stimulator for both the VNS and TNS stimulation sites. In some examples, the device is configured for TNS and VNS to be independently controlled to be active in different phases of treatment and recovery, such as delivery of TNS during one phase of treatment and the delivery of VNS during a second different phase of treatment. For example, TNS may be delivered during the first 24 hours of treatment, while VNS may be delivered during the first 48 hours of treatment. These hours of delivery are only for example, and TNS and VNS may be delivered for shorter or longer periods of time during their respective phases. Thus, the device may deliver TNS during one time period and deliver VNS during another time period and these time periods may be different (partially overlapping or not overlapping).

Controller 28 may be a relatively small device that may be secured to patient 14, for example, to the chest of patient 14 via one or more straps, tape, or the like. Controller 28 may be coupled to wires 50 which may electrically connect a stimulation generator in controller 28 to electrodes 51A-51N (collectively “electrodes 51”) of a device, such as patch 48, a headband, an eyemask, or the like. Patch 48 may be secured to a head of patient 14 via adhesive, a head band, or other securing mechanism. In this manner, an electrical stimulation signal may be generated by controller 28 according to one or more stimulation parameters and delivered to electrode 51 of patch 48 to provide TNS for patient 14. In some examples, patch 48 may be placed such that the TNS is delivered to ophthalmic zone 40 (FIG. 1). Such TNS may increase cerebral blood flow in patient 14. In some examples, patch 48 may include one or more sensors, such as sensor 49, which may sense one or more physiological parameters of patient 14.

Controller 28 may also be electrically coupled to an elongated member 30 of a transesophageal neurostimulation device via wires 47. The transesophageal neurostimulation device may include electrodes 34A-34N (collectively “electrodes 34”). Electrodes 34 may be electrically coupled to wires 47. In this manner, an electrical stimulation signal may be generated by controller 28 according to one or more stimulation parameters and delivered to electrodes 34 to provide transesophageal VNS for patient 14, for example, to anterior branch 26A and/or posterior branch 26B of the vagus nerve.

For example, a distal end of the transesophageal neurostimulation device of multi-site neurostimulation system 10 may be introduced into esophagus 24 through either nasal cavity 16 (as shown) or mouth 12 and may stimulate the vagus nerve through the wall of esophagus 24. One possible location of the delivery of stimulus would be at or near where 24 esophagus passes through a diaphragm, or caudal from the diaphragm (not shown), of patient 14. At this location, the vagus nerve is primarily organized into anterior branch 26A and posterior branch 26B (FIG. 2) that are both attached to the outer layer of esophagus 24. Esophagus 24 may be thin, around only about 1-3 mm thick, and even thinner if esophagus 24 is distended. Thus, multi-site neurostimulation system 10 may deliver electrical stimulation through the wall of esophagus 24 and to a portion of the vagus nerve. In some examples, multi-site neurostimulation system 10 may be used to stimulate one or more of branches of the vagus nerve, roots of the vagus nerve, ganglia of the vagus nerve, or plexus of the vagus nerve.

In the example of FIG. 3, multi-site neurostimulation system 10 includes controller 28, patch 48, elongated member 30, and expandable member 32. In some examples, elongated member 39 may be biased, such as being bent or weighted, in such a manner as to position electrodes 34 of multi-site neurostimulation system 10 at locations more likely to be near the vagus nerve, such as anterior branch 26A and/or posterior branch 26B. In some examples, multi-site neurostimulation system 10 may include a steerable or deflectable device configured to indent, appose, or penetrate electrodes of multi-site neurostimulation system 10 into an inner wall of esophagus 24. For example, the steerable or deflectable device may be flexible for insertion into esophagus 24 of patient 14, but include a bias to elongated member 30 and/or a direction of deflection that facilitates the positioning of electrodes 34 at locations more likely to be near the vagus nerve.

Esophagus 24 is located between the spinal column and the heart (neither shown in FIG. 2). The anterior of esophagus 24 is adjacent to the heart. In some examples, to reduce or avoid inadvertent heart stimulation, multi-site neurostimulation system 10 may be configured to direct stimulation towards posterior branch 26B or posterior trunk of the vagus nerve. For example, multi-site neurostimulation system 10 may include sensor 6 which may include an accelerometer which may be used to determine the posterior direction. For example, multi-site neurostimulation system 10 can detect the movements due to each heartbeat and determine the posterior direction being away from the detected acceleration. In addition, or alternatively, sensing electrodes of electrodes 34 or other electrodes, which may be located along elongated member 30, may be used to sense an electrocardiogram (ECG or EKG) of patient 14 and controller 28 may determine the posterior direction based on the sensed EKG signal. For example, EKG signals sensed from electrodes facing the posterior of patient 14 may sense a lower amplitude EKG then electrodes facing the anterior of patient 14. In some examples, elongated member 30 and/or expandable member 32 may be shaped in such a manner as to automatically orient the stimulation electrode(s) posteriorly. In some examples, sensing electrodes of electrodes 34 or other electrodes, which may be located along elongated member 30, may be used to sense an EKG of patient 14 to assist a clinician in otherwise positioning multi-site neurostimulation system 10. For example, controller 28 may determine the position of electrodes within patient 14 based on the sensed amplitude of the EKG signals and may display a representation of the position of at least a portion of multi-site neurostimulation system 10 with respect to patient anatomy which a clinician may use to guide multi-site neurostimulation system 10 into a preferred position.

Controller 28 may be configured to control neurostimulation being delivered to the trigeminal nerve and the vagus nerve of patient 14. For example, controller 28 may include processing circuitry, telemetry circuitry, and memory. The telemetry circuitry may be configured for wireless or wired communication. Controller 28 may include stimulation circuitry configured to generate one or more stimulation signals. For example, the stimulation circuitry may generate a stimulation signal for use for both TNS and VNS, or may generate two different signals, one for TNS and another for VNS.

In some examples, controller 28 may include a clinician programmer or patient programmer. In some examples, controller 28 may be a device for inputting stimulation programs or stimulation parameters into multi-site neurostimulation system 10 for the generation of stimulation signal(s). In some examples, controller 28 may be a wearable communication device, with a therapy request input integrated into a key fob or a wristwatch, handheld computing device, smart phone, computer workstation, or networked computing device. Controller 28 may include a user interface that is configured to receive input from a user (e.g., patient 14, a caretaker, or a clinician). In some examples, the user interface includes, for example, a keypad and a display, which may for example, be a liquid crystal display (LCD) or light emitting diode (LED) display. In some examples, the user interface may include a turnable knob or a representation of a turnable knob. The keypad may take the form of an alphanumeric keypad or a reduced set of keys associated with particular functions. Controller 28 may additionally or alternatively include a peripheral pointing device, such as a mouse, via which a user may interact with the user interface. In some examples, a display of controller 28 may include a touch screen display, and a user may interact with controller 28 via the display.

A user, such as a clinician, a patient, or a caregiver, may also interact with controller 28 to communicate with multi-site neurostimulation system 10. Such a user may interact with controller 28 to retrieve physiological or diagnostic information from sensor(s) that may be located on or in a portion of multi-site neurostimulation system 10 that is intended to be within patient 14 during stimulation, from sensor 49 of patch 48, and/or sensor 8 which may be external to patient 14.

The user may also interact with controller 28 to program multi-site neurostimulation system 10, e.g., select values for the stimulation parameters with which multi-site neurostimulation system 10 generates and delivers stimulation and/or the other operational parameters of multi-site neurostimulation system 10, such as one or more stimulation parameters (e.g., pulse amplitude, pulse width, pulse frequency, pulse burst duration, electrode combination, etc.), user requested periods for stimulation or periods to prevent stimulation, or any other such user customization of therapy.

For example, the user may use controller 28 to retrieve information from multi-site neurostimulation system 10 relating to a heartrate of patient 14, a heart rate variability over time, respiration rate, vagus nerve sensed activity, core body temperature, or the like. As another example, the user may use controller 28 to retrieve information from multi-site neurostimulation system 10 relating to the performance or integrity of multi-site neurostimulation system 10. In some examples, this information may be presented to the user as an alert if a system condition that may affect the efficacy of therapy is detected.

A user may, for example, use a keypad or touch screen of controller 28 to request multi-site neurostimulation system 10 to deliver or terminate the electrical stimulation. For example, patient 14 may use controller 28 to provide a therapy request to control the delivery of the electrical stimulation “on demand,” e.g., when patient 14 deems the second stimulation therapy desirable. This request may be a therapy trigger event used to terminate electrical stimulation. In some examples, a user may independently control the delivery of TNS and VNS. For example, a user may control via controller 28 the delivery of TNS separately from the delivery of VNS, such that multi-site neurostimulation system 10 may deliver TNS when not delivering VNS, may deliver VNS when not delivering TNS, or may deliver or not deliver both VNS and TNS simultaneously. Additionally, a user may independently control the stimulation parameters delivered by multi-site neurostimulation system 10, such that the same or different stimulation parameters may be used by the stimulation generator of controller 28 to generate stimulation programs for the delivery of TNS and/or VNS.

Controller 28 may provide a notification to patient 14 or a clinician when the electrical stimulation is being delivered or notify patient 14 of the prospective termination of the electrical stimulation. In such examples, controller 28 may display a visible message on a display device, emit an audible alert signal or provide a somatosensory alert (e.g., by causing a housing of controller 28 to vibrate). In other examples, the notification may indicate when therapy is available (e.g., a countdown in minutes, or indication that therapy is ready).

As patients in a hospital environment may need magnetic resonance imagery (MRI) to be taken by an MRI device or other procedures by other devices, controller 28 may be detachable or detached from elongated member 30 and patch 48 to facilitate the transportation of patient 14 to the device, insertion of patient 14 into the device, and operation of the device, if necessary. As such controller 28 may be detachable or separate from elongated member 30 and/or patch 48. Alternatively, multi-site neurostimulation system 10 may be MRI compatible such that multi-site neurostimulation system 10 does not substantially interfere with the images taken by the MRI device.

Electrodes 51 of patch 48 may be configured to be disposed separately from each other on patch 48. In some examples, multi-site neurostimulation system 10 may be configured to deliver a stimulation signal to the trigeminal nerve of patient 14 via electrodes 51 in a cycled manner. For example, the delivery of the stimulation signal may move over time between different electrode combinations of electrodes 51, such as delivering stimulation via electrode 51A and electrode 51B, then delivering stimulation via electrode 51B and another electrode, and so on. In this manner, a clinician may not need to align any particular electrodes of electrodes 51 with branches of the vagus nerve. In some examples, electrodes 51 may operate in a bipolar or multi-polar configuration. For example, one or more electrodes of electrodes 51 may be configured as anodes and one or more of electrodes 51 may be configured as cathodes. Such a configuration is different than a unipolar configuration which would include an electrode located at a position relatively remote from the trigeminal nerve. In other examples, electrodes 51 may operate in unipolar configuration. In such a case, the return electrode(s) may be located distant from patch 48, such as on a return pad on the skin of the patient. The return pad on the skin may be placed on the head, for example, near the ear, jaw, nose, or other portion of the head, to steer the current on a path the goes through the trigeminal nerve.

In some examples, two or more of electrodes 51 may be used to measure an impedance of tissue to determine a location of a target stimulation location, such as branches of the trigeminal nerve or a zone, such as ophthalmic zone 40 and to indicate if the electrodes are in good contact with the tissue. To measure the impedance of tissue, multi-site neurostimulation system 10 may source an electrical signal, such as current, to one electrode of electrodes 51, while another electrode of electrodes 51 sinks the electrical signal. Multi-site neurostimulation system 10 may then determine the voltage between these two electrodes. Multi-site neurostimulation system 10 may then determine the impedance of the tissue between the electrodes using a known value of the electrical signal sourced the determined voltage. In some examples, multi-site neurostimulation system 10 may be configured to detect branches of the trigeminal nerve or zones, such as through the use of sensed impedances, and select the desired branch(es) or zone(s) to stimulate. Multi-site neurostimulation system 10 may also be configured to select the appropriate electrode combination of electrodes 51 and/or other stimulation parameters to stimulate the desired branch(es) or zone(s).

Elongated member 30 may include conductors (not shown in FIG. 3) configured to conduct the stimulation signal from the stimulation circuitry of controller 28 to electrodes 34. Electrodes 34 may include two or more electrodes. In some examples, each of electrodes 34 may not form a closed loop so as reduce the risk of entanglement with another nasogastric tube, should another nasogastric tube be introduced or be already introduced into esophagus 24. Elongated member 30 may also define a lumen configured to permit the removal or introduction of substances from patient 14. For example, the lumen may permit the introduction of food, drink, medication, or the like from external of the patient into esophagus 24 or stomach 25.

Electrodes 34 may be disposed on expandable member 32. Expandable member 32 may be configured to expand from a non-expanded or collapsed state to a size approximately equal to the circumference of an internal wall of esophagus 24 thereby causing electrodes 34 to make physical contact with the internal wall of esophagus 24. In some examples, expandable member 32 may be configured to expand to distend the internal wall of esophagus 24. In some examples, expandable member 32 may include a balloon or other expandable structure, such as a mechanically expandable structure that includes struts and/or linkages that enables expansion (e.g., similar to a stent or cage).

In some examples, an additional expandable member 36 may be included in multi-site neurostimulation system 10. Expandable member 36 may be configured to expand in stomach 25 of patient 14, under the control of a clinician, in such a manner as to position electrodes 34 in a position to stimulate a target location in patient 14, such as one or more branches of the vagus nerve. For example, a clinician may desire to position electrodes 34 relative to a lower esophageal sphincter separating esophagus 24 from stomach 25. For example, esophagus 24 may be relatively thick and muscular at the lower esophageal sphincter, so it may be desirable to stimulate the vagus nerve cranially of the lower esophageal sphincter. In some examples, multi-site neurostimulation system 10 may be configured such that a distance between a proximal shoulder of expandable member 36 to electrodes 34 is of such a size that electrodes 34 are positioned proximal of the lower esophageal sphincter. In some examples, multi-site neurostimulation system 10 may be configured such that electrodes 34 are located in the range of about 2 cm to about 12 cm from the z-line of patient 14. The z-line is a term for a faint zig-zag impression at the gastro-esophageal junction. This impression demarcates the transition between the stratified squamous epithelium in the esophagus and the intestinal epithelium of the gastric cardia (e.g., the squamocolumnar junction). In some examples, expandable member 36 may include a balloon or other expandable structure, such as a mechanically expandable structure that includes struts and/or linkages that enables expansion (e.g., similar to a stent or cage).

In some examples, system 10 may include sensor 7 which may be positioned on expandable member 36, or in the example where system 10 does not include an expandable member 36, at or near a distal end of elongated member 30 (for example, distal to expandable member 32). Sensor 7 may be configured to generate a signal indicative the entry of sensor 7 into stomach 25 or entry into the lower esophageal sphincter of patient 14. In this manner, electrodes 34 (or other stimulation device) may be placed at an appropriate location for stimulation within esophagus 24 of patient 14 without a need to use fluoroscopy. For example, sensor 7 may include a pressure sensor. A pressure of esophagus 24, stomach 25, and the lower esophageal sphincter may be characteristically different. In this manner, controller 28 may determine when sensor 7 enters into stomach 25 or into the lower esophageal sphincter of patient 14. Sensor 7 may include a pH sensor. Sensor 7 may generate a signal indicative of a pH in patient 14. For example, the pH of esophagus 24 may typically be around 7.0, while a pH of stomach 25 may typically be in the range of 1.5 to 3.5. In this manner controller 28 may determine when sensor 7, and the distal portion of elongated member 30, enters into stomach 25. In some examples, based on the controller 28 determining that sensor 7 has entered into stomach 25, controller 28 may initiate stimulation and/or expansion of expandable member(s) 32 and/or 36.

In some examples, elongated member 30 and expandable members 32 and/or 36 may be sized in the range of from 8 to 18 French when expandable members 32 and/or 36 are in a non-expanded or collapsed state to enable relatively easy introduction of elongated member 30 and expandable members 32 and/or 36 within nasal cavity 16 or mouth 12 of patient 14. In some examples, in order to reduce sliding friction between system 10 and patient 14 during insertion of a portion of system 10 into patient 14, elongated member 30, expandable member 32, and/or expandable member 36 may be lubricated. In some examples, the lubrication may be contained within packaging that encloses at least a portion of system 10, may be pre-lubricated, or, in some examples, system 10 may be configured to self-lubricate. For example, controller 28 may include a lubrication pump that pump lubricant onto an exterior surface of elongated member 30, expandable member 32, and/or expandable member 36. In some examples, elongated member 30, expandable member 32, and/or expandable member 36 may define a lubricating lumen which may carry lubricant from the lubrication pump to an exterior surface of elongated member 30, expandable member 32, and/or expandable member 36 via lubrication openings. In other examples, a lubrication lumen of elongated member 30, expandable member 32, and/or expandable member 36 may be prefilled with lubricant and the pressure exerted upon elongated member 30, expandable member 32, and/or expandable member 36 by esophagus 24 during insertion of elongated member 30 into esophagus 24 may cause the prefilled lubricant to be discharged via the lubrication openings to the exterior surface of elongated member 30. In other examples, a coating may be applied to elongated member 30, expandable member 32, and/or expandable member 36 which may become lubricious when in contact with saliva or mucus of patient 14.

In some examples, in addition to, or alternatively, in order to reduce patient discomfort caused by system 10, elongated member 30, expandable member 32, and/or expandable member 36 may be pre-coated with a local anesthetic such as lidocaine. In some examples, the local anesthetic may be included in the packaging that encloses at least a portion of system 10. In some examples, the local anesthetic may be combined with a lubricant.

Multi-site neurostimulation system 10 may deliver electrical stimulation to patient 14 by generating and delivering a programmable electrical stimulation signal (e.g., in the form of electrical pulses or an electrical waveform) to a target a therapy site near electrodes 34 disposed, in some examples, on an outer surface of expandable member 32 and/or near electrodes 51 of patch 48. Elongated member 30, expandable member 32, and expandable member 36 may be constructed of biocompatible materials.

Electrodes 34 may be configured to be circumferentially separated from each other on an outer surface of expandable member 32. In some examples, multi-site neurostimulation system 10 may be configured to deliver a stimulation signal to the vagus nerve of patient 14 via electrodes 34 in a cycled manner. For example, the delivery of the stimulation signal may change, or alternate, over time between different electrode combinations of electrodes 34, such as delivering stimulation via electrode 34A and electrode 34B, then delivering stimulation via electrode 34B and another electrode, and so on. In this manner, a clinician may not need to circumferentially align any particular electrodes of electrodes 34 with branches of the vagus nerve. In some examples, electrodes 34 may operate in a bipolar or multi-polar configuration. For example, one or more electrodes of electrodes 34 may be configured as anodes and one or more of electrodes 34 may be configured as cathodes. Such a configuration is different than a unipolar configuration which would include an electrode located at a position relatively remote from the vagus nerve. In other examples, electrodes 34 may operate in unipolar configuration. In such a case, the return electrode(s) may be located on a portion of elongated member 30 in esophagus 24, distant from expandable member 32 (e.g., return electrode 37), or a return pad on the skin of the patient. The return pad on the skin may be placed on the abdomen near the lower esophagus to steer the current on a path the goes through the vagus nerve.

While electrodes 34 are depicted arranged in an array circumferentially separated from each other, the illustrated numbers and configurations of electrodes 34 are merely exemplary. Other configurations, e.g., numbers and positions of electrodes, are also contemplated. In some examples, the electrodes may be used for delivering different stimulation therapies or other electrical stimulations to respective stimulation sites within patient 14 or for monitoring at least one physiological marker of patient 14. For example, a set of electrodes may deliver stimulation at a first frequency to a first branch of the vagus nerve while a different set of electrodes may deliver stimulation at a second frequency to a second branch of the vagus nerve. In another example, a set of electrodes may deliver stimulation at a first frequency to a first location of first branch of the vagus nerve while a different set of electrodes may deliver stimulation at a second frequency to a second location of the first branch of the vagus nerve. This may allow for directional stimulation, such as blocking in a distal direction and stimulating in a proximal direction for an afferent stimulation. In some examples, the first frequency may be on the order of 1 Hz to 200 Hz for delivery of therapy (e.g., about 20 Hz) and the second frequency may be on the order of 1 kHz to 50 kHz for creating a nerve block (e.g., between about 10 kHz to about 20 kHz). In some examples, there may be separate electrodes of electrodes 34 for delivering blocking and stimulating, and these separate electrodes may be arranged along a transesophageal neurostimulation device rather than, or in addition to, circumferentially around the device.

In some examples, two or more of electrodes 34 may be used to measure an impedance of tissue to determine a location of a target stimulation location, such as branches of the vagus nerve and to indicate if the electrodes are in good contact with the tissue. To measure the impedance of tissue, multi-site neurostimulation system 10 may source an electrical signal, such as current, to one electrode of electrodes 34, while another electrode of electrodes 34 sinks the electrical signal. Multi-site neurostimulation system 10 may then determine the voltage between these two electrodes. Multi-site neurostimulation system 10 may then determine the impedance of the tissue between the electrodes using a known value of the electrical signal sourced the determined voltage. For example, there may be different effects of stimulating the anterior and posterior branches of the vagus nerve. Therefore, it may be desirable to selectively stimulate both branches, or only a specific branch of the vagus nerve, such as only the posterior branch to avoid stimulating the heart. In some examples, multi-site neurostimulation system 10may be configured to detect branches of the vagus nerve, such as through the use of sensed impedances, and select the desired branch(es) to stimulate. Multi-site neurostimulation system 10 may also be configured to select the appropriate electrode combination of electrodes 34 and/or other stimulation parameters to stimulate the desired branch(es).

In some examples, multi-site neurostimulation system 10 may include sensors, such as sensor 49 of patch 48, sensor 6 shown disposed on the surface of expandable member 32, or other sensors, such as sensor 8, which may monitor one or more physiological parameters of patient 14. In some examples, sensors 49, 6, and/or 8 may be configured to monitor vital signs of patient 14 such as an EKG. Controller 28 may monitor the vital signs of patient 14 based on signals from sensors 49, 6, and/or 8 and provide an alarm or alert based on such monitoring when the vital signs depart from a predetermined range by more than a predetermined amount.

In some examples, multi-site neurostimulation system 10 may change stimulation parameters, terminate stimulation, or initiate stimulation, of either TNS, VNS, or both TNS and VNS based on the sensed parameter(s). Such parameters may include heart rate, heart rate variability, respiration rate, vagus nerve sensed activity, core body temperature (or a surrogate therefor), electromyography (EMG), activity level of patient 14 (e.g., based on one or more accelerometer signals), pH of stomach 25 or esophagus 24, pressure in stomach 25 or esophagus 24, other physiological parameters of patient 14, and/or a patient indication of discomfort or pain. In this manner, multi-site neurostimulation system 10 may be configured to operate as a closed-loop system using data from one or more sensors to adjust delivered electrical stimulation.

In some examples, multi-site neurostimulation system 10 may automatically begin to deliver VNS with minimal user input. For example, multi-site neurostimulation system 10 may start stimulation in response to determining that a measured impedance is below a predetermined threshold, when expandable member 32 or expandable member 36 is inflated or otherwise expanded, or when a signal from sensor 7 is indicative of sensor 7 being in stomach 25 of patient 14.

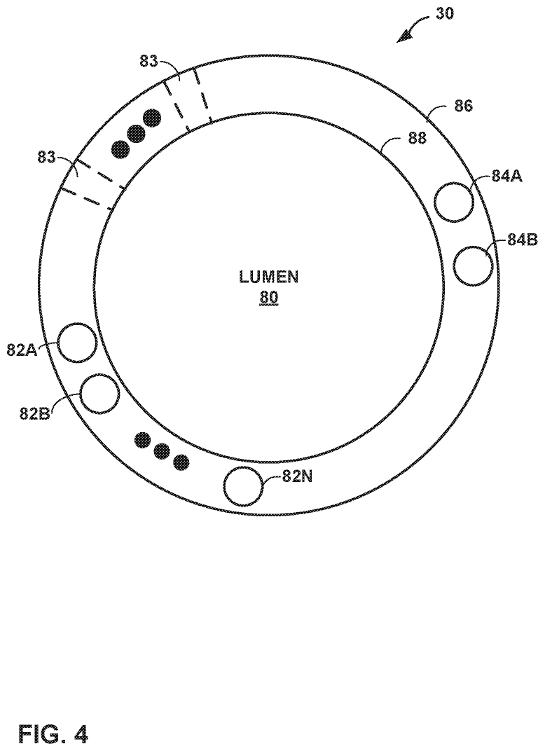

FIG. 4 is a conceptual diagram illustrating an example cross section of elongated member according to the techniques of this disclosure. In the example of FIG. 4, elongated member 30 includes an outer wall 86 and an inner wall 88 defining lumen 80. Lumen 80 may be configured to facilitate the introduction or removal of substances, such as food, fluids, medication, air, or the like, from esophagus 24 or stomach 25 of patient 14. For example, a clinician may use lumen 80 to inject or aspirate substances into or from stomach 25. Lumen 80 may be fluidically coupled to one or more fluid openings 83 near a distal end of elongated member 30 for injection or aspirating such substances. The proximal end of lumen 80 may include one or more other fluid openings for injecting or aspirating such substances. For example, a clinician may inject medication into the fluid opening(s) on the proximal end of lumen 80 and such medication may flow through lumen 80 to fluid opening(s) 83 and into stomach 25 of patient 14.

Elongated member 30 may also include one or more electrical conductors, such as conductors 82A, 82B, 82N, etc. (referred to hereinafter collectively as conductors 82). For example, elongated member 30 may include a conductor communicatively coupled to each of electrodes 34 disposed on expandable member 32 (FIG. 3). In some examples, electrical conductors 82 may each be configured to conduct electrical signals, such as a stimulation signal or a sensed signal between controller 28 and an associated electrode of electrodes 34.

Elongated member 30 may also define lumen 84A and/or lumen 84B. Lumen 84A may be configured to carry a substance or to house a mechanism for expanding expandable member 32 or expandable member 36. Lumen 84B may be configured to be coupled to an external inflation source and carry a substance, or to house a mechanism for expanding expandable member 36. For example, the substance may include air, saline, or any other gas or liquid which may be capable of inflating a balloon in examples where expandable members 32 or 36 include balloons and elongated member 30 may include at least one fluid opening for injecting or removing such substance which may be fluidically coupled to lumen 84A and/or lumen 84B. In examples in which expandable members 32 or 36 do not include balloons, lumen 84A and/or lumen 84B may be configured to receive a deployment mechanism (e.g., a pull wire or a push wire) for deploying an expandable structure. In such a case, elongated member 30 may include an access opening to provide the clinician with access to the deployment mechanism. In some examples, rather than elongated member 30 defining lumen 84A and/or 54B, a distal portion of elongated member 30 and expandable members 32 or 36 may be configured to be contained within a removeable sheath that may be removed by a clinician after a portion of elongated member 30 is inserted into esophagus 24 which may cause any expandable members to expand.

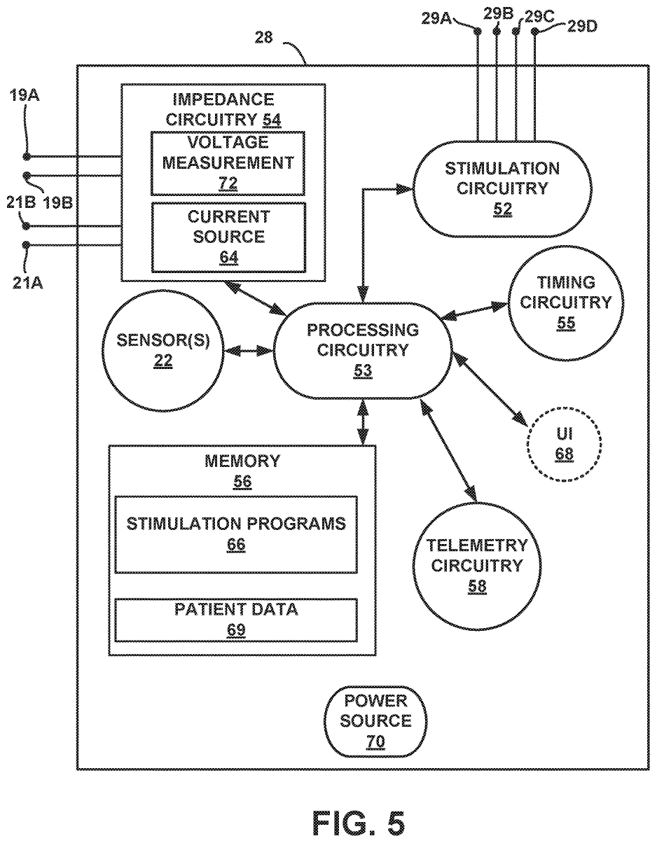

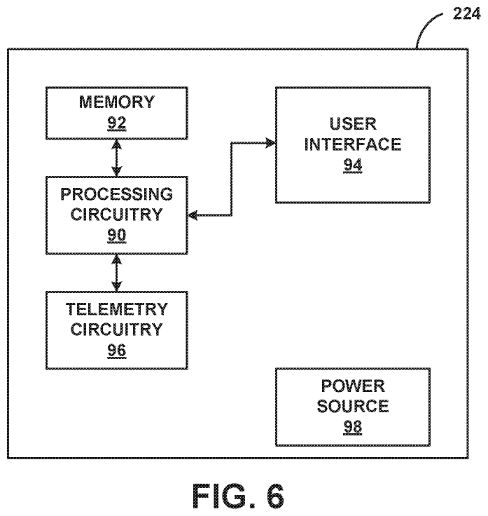

FIG. 5 is a block diagram of an example controller for a multi-site neurostimulation system according to one or more aspects of this disclosure. Controller 28 may include stimulation circuitry 52 configured to generate one or more stimulation signals, processing circuitry 53, telemetry circuitry 58, timing circuitry 55, memory 56, sensor(s) 22 (which may be an example any of sensors 49, and/or 6, 8 (FIG. 3) and impedance circuitry 54. Controller 28 may also include one or more electrodes, such as electrodes 29A-29D (collectively referred to hereinafter as electrodes 29), electrodes 19A-19B, and electrodes 21A-21B. At least a portion of electrodes 29, 19A-19B, and 21A-21B, may be examples of electrodes 51 (FIG. 3). At least a portion of electrodes 29, 19A-19B, and 21A-21B, may be examples of electrodes 34 (FIG. 3). For example, electrodes 29A-29B may represent electrodes 51, while electrodes 29C-29D may represent electrodes 34. It should be noted that while a specific number of electrodes 19, 21, and 29 are shown in FIG. 5, different numbers of electrodes are contemplated. For example, there may be four electrodes 19, four electrodes 21, and eight electrodes 29. In some examples, controller 28 may also include a user interface (UI 68) which may function similarly to user interface 94 of FIG. 6 described in more detail in the discussion of FIG. 6 below.

Sensor(s) 22 may comprise a patient motion sensor that generates a signal indicative of patient posture state, orientation, or activity level. In some examples, controller 28 may use sensor(s) 22 (which may include an accelerometer) to identify posture states of patient 14. Processing circuitry 53 may use the posture state to determine a position of one or more electrodes 29 and may use the position to determine which electrode combination or other stimulation parameters to use for stimulation, for example. For example, stimulation programs 66 may include predetermined programs for supine, prone, lateral, or other common surgical positions. By knowing the posture state, the likely position of the vagus branches on patient 14, and the orientation of the electrodes of controller 28, processing circuitry 53 may automatically select the electrodes to be used for stimulation. Controller 28 may also operate in a closed-loop manner by controlling stimulation parameters and the delivery of stimulation is response to sensed physiologic parameters sensed by sensor(s) 22, such as heart rate, heart rate variability, respiration rate, trigeminal nerve sensed activity, vagus nerve sensed activity, core body temperature (or a surrogate therefor), EMG, activity level of patient 14, or other measures. These physiological parameters may be sensed by sensor(s) 22 and/or impedance circuitry 54. For example, processing circuitry 53 may control stimulation circuitry 52 to titrate and optimize the neurostimulation therapy based on the sensed physiological parameters. For example, processing circuitry 53 may monitor heart rate variability of patient 14 and, when the heart rate variability meets a heart rate variability threshold, terminate therapy.

In some examples, sensor(s) 22 may include one or more thermocouples. For example, after a stroke a core body temperature of patient 14 may rise, peak, and then come back down. In some examples, processing circuitry 53 may monitor the sensed core body temperatures and, after the sensed core body temperatures (e.g., each of the sensed core body temperatures, an average of the sensed core body temperatures, or the like) peak and based on the sensed core body temperatures reaching a threshold temperature, automatically change or terminate stimulation.

In some examples, sensor(s) 22 may include one or more pressure sensors, which may be located external to patient 14 or may be located along elongated member 30, on expandable member 32, on expandable member 36, and/or on patch 48. Such pressure sensor(s) may be used to monitor peristaltic pressure waves, gastric pressure, blood pressure, or the like.

In some examples, the neurostimulation could be delivered to the trigeminal nerve (in ophthalmic zone 40, maxillary zone 42, mandibular zone 44, and/or trigeminal nerve ganglion 38) and/or the vagus nerve (in neck, chest, or abdomen). While the target tissue for the delivery of stimulation is primarily discussed herein as being the trigeminal nerve and the vagus nerve, other potential locations of interest may include the sacral nerve, the pudendal nerve, the splenic nerve, the splanchnic nerve, tibial nerve, or other peripheral nerves.

In some examples, the physiological parameters may be sensed by external devices, such as pulse oximetry sensors, Near Infrared Spectroscopy(NIRS), Bispectral Index processed electroencephalogram (EEG), EMG electrodes, EEG electrodes, wearable activity tracker, cameras, depth-sensing cameras, or other sensors. In some examples, physiological parameters may be measured by anesthesia equipment such as a multi-parameter monitor (MPM) or respirator. In some examples, the physiological parameters may be sensed by an implantable sensor such as in a pacemaker or cardiac monitor. By using sensed physiological parameters to control the stimulation, processing circuitry 53 may maximize, optimize, or otherwise improve the stimulation of the CAP and the stimulation of the trigeminal nerve.

In some examples, processing circuitry 53 may utilize the sensed physiological parameters to provide feedback to a clinician indicative of whether the trigeminal and/or vagus nerve is actually being stimulated.

According to some examples, processing circuitry 53 identifies changes to the patient's physiological state that are relevant to desired changes in neurostimulation. For example, processing circuitry 53 may control stimulation circuitry 52 to generate a stimulation signal that is gated to the respiratory cycle or heartbeat. VNS may be more effective when gated to certain physiological activities. For example, it may enhance the potency of the VNS if the stimulation is gated to be during a phase of respiration, such as the exhalation phase of respiration. For example, the respiration cycle of patient 14 may be accurately detected with pulse oximetry signal analysis or an accelerometer in the device. In some examples, processing circuitry 53 may use other physiologic activities to gate the stimulation. For example, processing circuitry 53 may determine heart rate or circadian rhythms and gate the stimulation signal based on the heart rate, phase of a cardiac cycle, or a phase of a circadian rhythm. In some examples, processing circuitry 53 may similarly gate TNS.

Monitoring other physiological parameters may also serve to enhance safety. For example, stimulating the cervical vagus may depress the heart rate of patient 14. Processing circuitry 53 may be configured to control stimulation circuitry 52 to stop stimulation or lower a stimulation intensity if the heart declined below a threshold. Similarly, processing circuitry 53 may monitor sensed vital signs to monitor pain in an unconscious person. Processing circuitry 53 may be configured to control stimulation circuitry 52 to stop stimulation or lower a stimulation intensity if processing circuitry 53 determines that increasing pain is not associated with surgery or changes in anesthesia. In some examples, processing circuitry 53 may use one or more of the sensed parameters to balance between a parasympathatic and sympathetic tone in patient 14.

In general, controller 28 may comprise any suitable arrangement of hardware, alone or in combination with software and/or firmware, to perform the techniques attributed to controller 28 and processing circuitry 53, stimulation circuitry 52, impedance circuitry 54, and telemetry circuitry 58 of controller 28. In various examples, controller 28 may include one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components. Controller 28 also, in various examples, may include a memory 56, such as random access memory (RAM), read only memory (ROM), programmable read only memory (PROM), erasable programmable read only memory (EPROM), electronically erasable programmable read only memory (EEPROM), flash memory, comprising executable instructions for causing the one or more processors to perform the actions attributed to them. Moreover, although processing circuitry 53, stimulation circuitry 52, impedance circuitry 54, and telemetry circuitry 58 are described as separate circuitry, in some examples, processing circuitry 53, stimulation circuitry 52, impedance circuitry 54, and telemetry circuitry 58 are functionally integrated. In some examples, processing circuitry 53, stimulation circuitry 52, impedance circuitry 54, and telemetry circuitry 58 correspond to individual hardware units, such as microprocessors, ASICs, DSPs, FPGAs, or other hardware units. In further examples, any of processing circuitry 53, stimulation circuitry 52, impedance circuitry 54, and telemetry circuitry 58 may correspond to multiple individual hardware units such as microprocessors, ASICs, DSPs, FPGAs, or other hardware units.

Memory 56 stores stimulation programs 66 that specify stimulation parameter values for the electrical stimulation provided by controller 28. Stimulation programs 66 may also store information relating to determining and using physiological parameters, such as threshold values. In some examples, controller 28 may deliver stimulation therapy based on one or more physiological markers. In other examples, controller 28 may deliver stimulation therapy that is not based on one or more physiological markers. In some examples, memory 56 also stores patient data 69 which may include sensed physiological parameters. Patient data 69 may also include timing information which may be associated with the sensed physiological parameters.

Generally, stimulation circuitry 52 generates and delivers electrical stimulation under the control of processing circuitry 53. For example, processing circuitry 53 may control stimulation circuitry 52 to generate both a first stimulation signal (for transcutaneous or percutaneous stimulation) and a second stimulation signal (for transesophageal stimulation) simultaneously. For example, stimulation circuitry 52 may be a single circuitry or unit. In some examples, processing circuitry 53 controls stimulation circuitry 52 by accessing memory 56 to selectively access and load at least one of stimulation programs 66 to stimulation circuitry 52. For example, in operation, processing circuitry 53 may access memory 56 to load one of stimulation programs 66 to stimulation circuitry 52. In other examples, stimulation circuitry 52 may access memory 56 and load one of the stimulation programs 66. In some examples, the electrical stimulation signal generated and delivered by stimulation circuitry 52 may be above around 10 Hz to avoid activating muscular contraction.

In some examples, stimulation programs 66 may include stimulation programs that are configured to stimulate different nerves, such as a trigeminal nerve and a vagus nerve. In some examples, stimulation programs 66 may include stimulation programs that are configured to facilitate different effects. For example, stimulation circuitry may use different stimulation programs to generate different electrical stimulation signals to cause different effects. In some examples, stimulation circuitry 52 may generate an electrical stimulation signal in the range of about 1 to 200 Hz to reduce inflammation in patient 14 (e.g., around 20 Hz) or generate an electrical stimulation signal in the range of about 1 kHz to about 50 kHz to block and increase an inflammatory response (e.g., between about 10 kHz to about 20 kHz).

By way of example, processing circuitry 53 may access memory 56 to load one or more of stimulation programs 66 to stimulation circuitry 52 for delivering the electrical stimulation to patient 14. A clinician or patient 14 may select one or more particular programs of stimulation programs 66 from a list using a programming device, such as controller 28 (FIG. 5). Additionally, or alternatively, processing circuitry 53 may receive the selection via telemetry circuitry 58. Stimulation circuitry 52 delivers the electrical stimulation to patient 14 according to the selected program(s) for an extended period of time, such as minutes, hours, days, or until patient 14 or a clinician manually stops or changes the program.

Stimulation circuitry 52 delivers electrical stimulation (e.g., TNS and/or VNS) according to stimulation parameters. In some examples, stimulation circuitry 52 delivers electrical stimulation in the form of electrical pulses. In such examples, relevant stimulation parameters may include a voltage amplitude, a current amplitude, a pulse rate, a pulse width, a duty cycle, a duty cycle of the stimulation ON/OFF periods, or the combination of electrodes 29 that stimulation circuitry 52 uses to deliver the stimulation signal. In other examples, stimulation circuitry 52 delivers electrical stimulation in the form of continuous waveforms. In such examples, relevant stimulation parameters may include a voltage or current amplitude, a frequency, a shape of the stimulation signal, a duty cycle of the stimulation signal, or the combination of electrodes 29 stimulation circuitry 52 uses to deliver the stimulation signal.

In the example illustrated in FIG. 4 impedance circuitry 54 includes voltage measurement circuitry 72 and current source 64, and may include an oscillator (not shown) or the like for producing an alternating signal. In some examples, impedance circuitry 54 may use a four-wire, or Kelvin, arrangement. As an example, processing circuitry 53 may periodically control current source 64 to, for example, source an electrical current signal through electrode 19A and sink the electrical current signal through electrode 21A. In some examples, for collection of impedance measurements, current source 64 may deliver electrical current signals that do not deliver stimulation therapy to the trigeminal nerve and/or vagus nerve, e.g., sub-threshold signals, due to, for example, the amplitudes or widths of such signals and/or the timing of delivery of such signals. Impedance circuitry 54 may also include a switching circuitry (not shown) for selectively coupling electrodes 19A, 19B, 21A, and 21B to current source 64 and voltage measurement circuitry 72. Voltage measurement circuitry 72 may measure the voltage between electrodes 19B and 21B. Voltage measurement circuitry 72 may include sample and hold circuitry or other suitable circuitry for measuring voltage amplitudes. Processing circuitry 53 may determine an impedance value from the measure voltage values received from voltage measurement circuitry 72.

In some examples, processing circuitry 53 may control stimulation circuitry 52 to deliver or terminate the electrical stimulation based on patient or clinician input received via telemetry circuitry 58. Telemetry circuitry 58 includes any suitable hardware, firmware, software or any combination thereof for communicating with another device, such as controller 28 (FIG. 5) or another device external to controller 28. Under the control of processing circuitry 53, telemetry circuitry 58 may receive communications, e.g., patient or clinician input, from and send communications, e.g., an alert, to controller 28. In the example, where controller 28 is representative of multi-site neurostimulation system 10C, controller 28 may use an antenna (not shown) when communicating, which may be internal and/or external. Processing circuitry 53 may provide the data to be sent to controller 28 and the control signals for the telemetry circuit within telemetry circuitry 58, and receive data from telemetry circuitry 58.

Generally, processing circuitry 53 may control telemetry circuitry 58 to exchange information with controller 28 or another device external to controller 28 wirelessly or wired. Processing circuitry 53 may transmit operational information and patient data 69 and receive stimulation programs or stimulation parameter adjustments via telemetry circuitry 58. Also, in some examples, controller 28 may communicate with other devices, such as stimulators, control devices, or sensors, via telemetry circuitry 58.

In some examples, power source 70 delivers operating power to the components of controller 28. In some examples, power source 70 may include a battery and a power generation circuit to produce the operating power. In some examples, the battery may be rechargeable to allow extended operation. Recharging may be accomplished through proximal inductive interaction between an external charger and an inductive charging coil within controller 28. In other examples, an external inductive power supply may power controller 28 whenever electrical stimulation is to occur. In some examples, power source 70 may be coupled to an external power source, such as an outlet on a hospital wall.

A stimulation program of stimulation programs 66 may define various parameters of the stimulation waveform(s) and electrode configuration(s) which result in a predetermined stimulation intensity being delivered to the targeted trigeminal nerve and/or vagus nerve. In some examples, the stimulation program(s) define parameters for at least one of a current or voltage amplitude of the stimulation signal(s), a frequency or pulse rate of the stimulation, the shape of the stimulation waveform, a duty cycle of the stimulation, a pulse width of the stimulation, a duty cycle of the stimulation ON/OFF periods, and/or the combination(s) of electrodes 34 and respective polarities of the subset of electrodes 34 used to deliver the stimulation. Together, these stimulation parameter values may be used to define the stimulation intensity (also referred to herein as a stimulation intensity level). In some examples, if stimulation pulses are delivered in bursts, a burst duty cycle also may contribute to stimulation intensity. Also, independent of intensity, a particular pulse width and/or pulse rate may be selected from a range suitable for causing the desired therapeutic effect after stimulation is terminated and, optionally, during stimulation. In addition, as described herein, a period during which stimulation is delivered may include on and off periods (e.g., a duty cycle or bursts of pulses) where even the short inter-pulse durations of time when pulses are not delivered are still considered part of the delivery of stimulation. A period during which controller 28 withholds stimulation delivery is a period in which no stimulation program is active for a given nerve and controller 28 is not tracking pulse durations or inter-pulse durations that occur as part of the electrical stimulation delivery scheme for such nerve). In addition to the above stimulation parameters, the stimulation may be defined by other characteristics, such as a time for which stimulation is delivered, a time for which stimulation is terminated, and times during which stimulation is withheld.

FIG. 6 is a block diagram illustrating an example configuration of a computing device in accordance with one or more aspects of this disclosure. Computing device 224 may include notebook computer, a smart phone, a workstation, a key fob, or a wearable device, for example. In some examples, computing device 224 may be configured to control one or more stimulation generators and/or sensors, such as transmitting instructions to controller 28. As illustrated in FIG. 6, computing device 224 may include a processing circuitry 90, memory 92, user interface 94, telemetry circuitry 96, and power source 98. Memory 92 may store program instructions that, when executed by processing circuitry 90, cause processing circuitry 90 and computing device 224 to provide the functionality ascribed to controller 28 throughout this disclosure. In general, computing device 224 comprises any suitable arrangement of hardware, alone or in combination with software and/or firmware, to perform the techniques attributed to computing device 224, and processing circuitry 90, user interface 94, and telemetry circuitry 96 of computing device 224.

In various examples, computing device 224 may include one or more processors, such as one or more microprocessors, DSPs, ASICs, FPGAs, or any other equivalent integrated or discrete logic circuitry, as well as any combinations of such components. Computing device 224 also, in various examples, may include a memory 92, such as RAM, ROM, PROM, EPROM, EEPROM, flash memory, a hard disk, a CD-ROM, comprising executable instructions for causing the one or more processors to perform the actions attributed to them. Moreover, although processing circuitry 90 and telemetry circuitry 96 are described as separate circuitry, in some examples, processing circuitry 90 and telemetry circuitry 96 are functionally integrated. In some examples, processing circuitry 90 and telemetry circuitry 96 and telemetry circuitry 58 correspond to individual hardware units, such as microprocessors, ASICs, DSPs, FPGAs, or other hardware units. In other examples, any of processing circuitry 90 and telemetry circuitry 96 and telemetry circuitry 58 may correspond to multiple individual hardware units, such as microprocessors, ASICs, DSPs, FPGAs, or other hardware units.

Memory 92 may store program instructions that, when executed by processing circuitry 90, cause processing circuitry 90 and computing device 224 to provide the functionality ascribed to computing device 224 throughout this disclosure. In some examples, memory 92 may further include program information, e.g., stimulation programs defining the neurostimulation, similar to those stored in memory 56 of controller 28. The stimulation programs stored in memory 92 may be downloaded into memory 56 of controller 28.

In certain examples, computing device 224 includes a user interface 94 that allows the for programming of the stimulation programs and for a patient to provide input. Patient 14 may, additionally or alternatively, request a change in stimulation program or settings through user interface 94.

User interface 94 may include a button or keypad, lights, a speaker for voice commands, a turnable knob, a display, such as a liquid crystal (LCD), light-emitting diode (LED), or cathode ray tube (CRT). In some examples the display may be a touch screen. As discussed in this disclosure, processing circuitry 90 may present and receive information relating to electrical stimulation and resulting therapeutic effects via user interface 94. For example, processing circuitry 90 may receive patient input via user interface 94. The input may be, for example, in the form of pressing a button on a keypad or selecting an icon from a touch screen. For example, patient 14 may provide input relating to a perception by patient 14 of discomfort or pain, such as the existence of discomfort or pain or a rating of discomfort or pain on a scale. In some examples, processing circuitry 90 may use such input to change stimulation parameters delivered by multi-site neurostimulation system 10, as discussed herein.

Processing circuitry 90 may also present information to the patient in the form of alerts related to delivery of the electrical stimulation to patient 14 or a caregiver via user interface 94. Although not shown, computing device 224 may additionally or alternatively include a data or network interface to another computing device, to facilitate communication with the other device, and presentation of information relating to the electrical stimulation and therapeutic effects after termination of the electrical stimulation via the other device.

Telemetry circuitry 96 supports wireless or wired communication between controller 28 and computing device 224 under the control of processing circuitry 90. Telemetry circuitry 96 may also be configured to communicate with another computing device via wireless communication techniques, or direct communication through a wired connection. In some examples, telemetry circuitry 96 may be substantially similar to telemetry circuitry 58 of controller 28 described above, providing wireless communication via an RF or proximal inductive medium. In some examples, telemetry circuitry 96 may include an antenna, which may take on a variety of forms, such as an internal or external antenna.

Examples of local wireless communication techniques that may be employed to facilitate communication between computing device 224 and another computing device include RF communication according to the 802.11 or Bluetooth specification sets, infrared communication, e.g., according to the IrDA standard, or other standard or proprietary telemetry protocols. In this manner, other external devices may be capable of communicating with computing device 224 without needing to establish a secure wireless connection.

Power source 98 delivers operating power to the components of computing device 224. Power source 98 may include a battery and a power generation circuit to produce the operating power. In some examples, the battery may be rechargeable to allow extended operation.

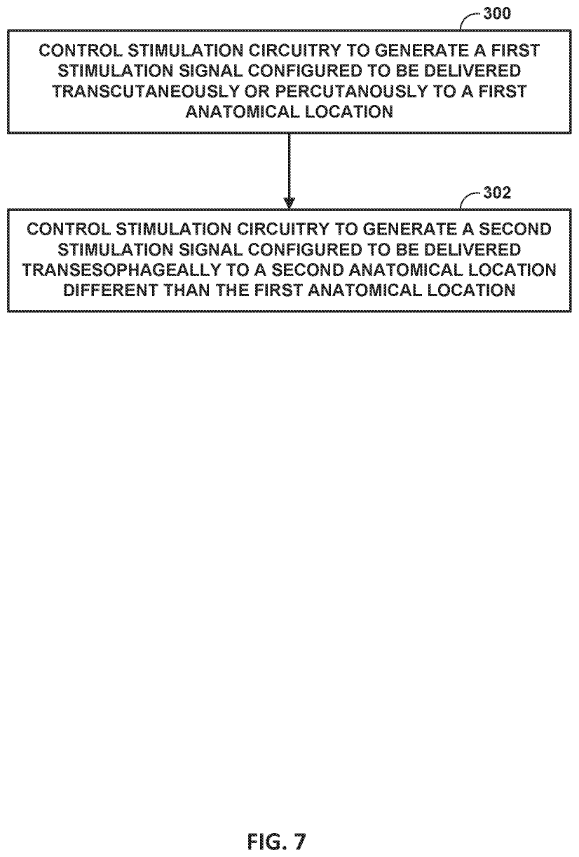

FIG. 7 is a flow diagram illustrating example multi-site neurostimulation techniques in accordance with one or more aspects of this disclosure. The example of FIG. 7 is described with respect to system 10, but any other devices or systems described herein may be used in other examples. Processing circuitry 53 may determine to start generating a first stimulation signal. Processing circuitry 53 may control stimulation circuitry 52 to generate a first stimulation signal configured to be delivered transcutanously or percutaneously to a first anatomical location (300). The first stimulation signal may be at least partially defined by first stimulation parameters of stimulation programs 66. For example, processing circuitry 53 may control stimulation circuitry 52 to generate the first stimulation signal which may be delivered by one or more of electrodes 51 to stimulate, transcutaneously or percutanously, a trigeminal nerve of patient 14. By being configured to be delivered transcutanously or percutaneously, the first stimulation signal may include or be based on stimulation parameters which may be efficacious for transcutaneous or percutaneous stimulation of a nerve, such as a trigeminal nerve.

Processing circuitry 53 may determine to generate a second stimulation signal. Processing circuitry 53 may control stimulation circuitry 52 to generate a second stimulation signal configured to be delivered transesophageally to a second anatomical location different than the first anatomical location (702). The second stimulation signal may be at least partially defined by second stimulation parameters of stimulation programs 66. For example, processing circuitry 53 may control stimulation circuitry 52 to generate the second stimulation signal which may be delivered by one or more of electrodes 34 to stimulate, transesophageally, a vagus nerve of patient 14. By being configured to be delivered transesophageally, the second stimulation signal may include or be based on stimulation parameters which may be efficacious for transesophageal stimulation of a nerve, such as a vagus nerve. Processing circuitry 53 may determine to stop generating at least one of the first stimulation signal or the second stimulation signal. Based on the determination to stop generating the at least one of the first stimulation signal or the second stimulation signal, processing circuitry 53 may control stimulation circuitry 52 to stop generating the at least one of the first stimulation signal or the second stimulation signal. If processing circuitry 53 controls stimulation circuity 52 to stop generating one of the first stimulation signal or the second stimulation signal, at a different time, processing circuitry 53 may determine to stop generating the other of the first stimulation signal or the second stimulation signal and control stimulation circuitry 52 to stop generating the other of the first stimulation signal or the second stimulation signal.

In some examples, multi-site stimulation system 10 includes stimulation circuitry 52 configured to generate a first stimulation signal and a second stimulation signal. In some examples, multi-site stimulation system 10 includes memory 56 configured to store first stimulation parameters that at least partially define the first stimulation signal and second stimulation parameters that at least partially defined the second stimulation signal (e.g., of stimulation programs 66). In some examples, multi-site stimulation system 10 includes processing circuitry 53 communicatively coupled to memory 56, and stimulation circuitry 52. In some examples, multi-site stimulation system 10 includes stimulation circuitry 52, memory 56, and processing circuitry 53 housed in a single device (e.g., controller 28). In some examples, processing circuitry 53 is configured to control stimulation circuitry 52 to generate the first stimulation signal configured to be delivered transcutaneously or percutaneously and control stimulation circuitry 52 to generate the second stimulation signal configured to be delivered transesophageally.

In some examples, processing circuitry 53 is configured to independently control stimulation circuitry 52 to generate the first stimulation signal and to generate the second stimulation signal. For example, processing circuitry 53 may be configured to control stimulation circuitry 52 to change one of the first stimulation signal or the second stimulation signal without changing another of the first stimulation signal or the second stimulation signal. For example, changing the first stimulation signal or the second stimulation signal may include changing at least one stimulation parameter of the one of the first stimulation signal or the second stimulation signal or ceasing the generation of the one of the first stimulation signal or the second stimulation signal.

In some examples, the first stimulation signal is different than the second stimulation signal. In some examples, processing circuitry 53 is configured to control stimulation circuitry 52 to generate the first stimulation signal during a first time period and control stimulation circuitry 52 to generate the second stimulation signal during a second time period, wherein the first time period and the second time period are different. In some examples, stimulation circuitry 52 is configured to simultaneously generate both the first stimulation signal and the second stimulation signal.

In some examples, multi-site neurostimulation system 10 includes a trigeminal neurostimulation (TNS) device (e.g., patch 48), the TNS device including at least one electrode (e.g., electrodes 51), the at least one electrode being configurable for delivery of the first stimulation signal to a trigeminal nerve of patient 14. In some examples, TNS device is configurable to deliver the first stimulation signal to the trigeminal nerve of patient 14 via at least one of ophthalmic zone 40, the maxillary zone 42, the mandibular zone 44, or trigeminal nerve ganglion 38.

In some examples, multi-site neurostimulation system 10 includes elongated member 30 configured to be at least partially inserted into esophagus 24 of patient 14 and expandable member 32 having a plurality of electrodes (e.g., electrodes 34) disposed on an outer surface, the plurality of electrodes being configurable for delivery of the second stimulation signal to a vagus nerve of the patient. In some examples, elongated member 30 is a nasogastric tube.

In some examples, multi-site neurostimulation system 10 includes one or more sensors (e.g., sensors 6-8 and/or 49), the one or more sensors being configured to sense one or more physiological parameters of patient 14. In some examples, processing circuitry 53 is further configured to control stimulation circuitry 52 based at least in part on the sensed one or more physiological parameters. For example, processing circuitry 53 may control stimulation circuitry 52 to start the generation of, stop the generation of, or change one or more of the first stimulation signal or the second stimulation signal in response to the sensed one or more physiological parameters.

In some examples, multi-site neurostimulation system 10 is configured to treat at least one of stroke, traumatic brain injury (TBI), or subarachnoid hemorrhage (SAH). System 10 may be configured and used to treat other injury or illness in other examples. In some examples, multi-site neurostimulation system 10 is a single device. For example, multi-site neurostimulation system 10 may be manufactured and/or packaged for sale as a single unit.

In some examples, a device includes stimulation circuitry 52 configured to generate a first stimulation signal and a second stimulation signal. In some examples, the device includes memory 56 configured to store first stimulation parameters that at least partially define the first stimulation signal and second stimulation parameters that at least partially defined the second stimulation signal. For example, memory 56 may store the first stimulation parameters and the second stimulation parameters in stimulation programs 66. In some examples, the device includes first electrodes (e.g., electrodes 51) configured to transcutaneously or percutaneously deliver the first stimulation signal to a first nerve of a patient. In some examples, the device includes second electrodes (e.g., electrodes 34) configured to transesophageally deliver the second stimulation signal to a second nerve of the patient. In some examples, the device includes processing circuitry 53 communicatively coupled to memory 56, and stimulation circuitry 52. In some examples, processing circuitry 53 is configured to control stimulation circuitry 52 to generate the first stimulation signal and control stimulation circuitry 52 to generate the second stimulation signal.

In some examples, the device includes patch 48 configured to be applied to skin of patient 14. In some examples, the first plurality of electrodes (e.g., electrodes 51) are disposed on patch 48. In some examples, the device includes a nasogastric tube (e.g., elongated member 30) configured to be at least partially inserted into esophagus 24 of patient 14. In some examples, the device includes expandable member 32 disposed on the nasogastric tube. In some examples, the second plurality of electrodes (e.g., electrodes 34) are disposed on expandable member 32.

In some examples, the first nerve is a trigeminal nerve and the second nerve is a vagus nerve.

The techniques of this disclosure may facilitate the stimulating the cervical, thoracic, or abdominal vagus branches in a manner that is relatively easy and quick to use, such as through transesophageal stimulation. Such techniques may be used for short-term stimulation, such as during an acute health problem, such as surgery or during an abrupt illnesses, such as sepsis, without having to undertake an invasive surgical procedure to implant a VNS device.

It should be noted that the techniques described herein, may not be limited to treatment or monitoring of a human patient. In alternative examples, the techniques of this disclosure may be applied to non-human patients, e.g., primates, canines, equines, pigs, and felines. These other animals may undergo clinical or research therapies that my benefit from the subject matter of this disclosure.

Various examples are discussed relative to one or more stimulation devices. It is recognized that the stimulation devices may include features and functionality in addition to electrical stimulation. Many of these additional features are expressly discussed herein. A few example features include, but are not limited to, different types of sensing capabilities and different types of wireless communication capabilities. For ease of discussion, the present disclosure does not expressly recite every conceivable combination of the additional features, such as by repeating every feature each time different examples and uses of the stimulation devices are discussed.

The techniques of this disclosure may be implemented in a wide variety of computing devices, medical devices, or any combination thereof. Any of the described units, circuitry or components may be implemented together or separately as discrete but interoperable logic devices. Depiction of different features as circuitry or units is intended to highlight different functional aspects and does not necessarily imply that such circuitry or units must be realized by separate hardware or software components. Rather, functionality associated with one or more circuitry or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various circuitry and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processing circuitry” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, circuitry or components may be implemented together or separately as discrete but interoperable logic devices. Depiction of different features as circuitry or units is intended to highlight different functional aspects and does not necessarily imply that such circuitry or units must be realized by separate hardware or software components. Rather, functionality associated with one or more circuitry or units may be performed by separate hardware or software components, or integrated within common or separate hardware or software components. For example, any circuitry described herein may include electrical circuitry configured to perform the features attributed to that particular circuitry, such as fixed function processing circuitry, programmable processing circuitry, or combinations thereof.

In some examples, a computer-readable storage medium comprises non-transitory medium. The term “non-transitory” may indicate that the storage medium is not embodied in a carrier wave or a propagated signal. In certain examples, a non-transitory storage medium may store data that may, over time, change (e.g., in RAM or cache).

This disclosure includes the following non-limiting examples.
Example 1. A system comprising: stimulation circuitry configured to generate a first stimulation signal and a second stimulation signal; memory configured to store first stimulation parameters that at least partially define the first stimulation signal and second stimulation parameters that at least partially defined the second stimulation signal; and processing circuitry communicatively coupled to the memory, and the stimulation circuitry, the processing circuitry being configured to: control the stimulation circuitry to generate the first stimulation signal configured to be delivered transcutaneously or percutaneously to a first anatomical location; and control the stimulation circuitry to generate the second stimulation signal configured to be delivered transesophageally to a second anatomical location different than the first anatomical location.
Example 2. The system of example 1, wherein processing circuitry is configured to independently control the stimulation circuitry to generate the first stimulation signal and to generate the second stimulation signal.
Example 3. The system of example 1 or 2, wherein the first stimulation signal is different than the second stimulation signal.
Example 4. The system of any of examples 1-3, wherein the processing circuitry is configured to control the stimulation circuitry to generate the first stimulation signal during a first time period; and control the stimulation circuitry to generate the second stimulation signal during a second time period, wherein the first time period and the second time period are different.
Example 5. The system of any of examples 1-4, wherein the stimulation circuitry is configured to simultaneously generate both the first stimulation signal and the second stimulation signal.
Example 6. The system of any of examples 1-5, further comprising a trigeminal neurostimulation (TNS) device, the TNS device comprising at least one electrode, the at least one electrode being configurable for delivery of the first stimulation signal to a trigeminal nerve of a patient.
Example 7. The system of example 6, wherein the TNS device is configurable to deliver the first stimulation signal to the trigeminal nerve of the patient via at least one of an ophthalmic zone, a maxillary zone, a mandibular zone, or a trigeminal nerve ganglion.
Example 8. The system of any of examples 1-7, further comprising: an elongated member configured to be at least partially inserted into an esophagus of a patient; and an expandable member having a plurality of electrodes disposed on an outer surface, the plurality of electrodes being configurable for delivery of the second stimulation signal to a vagus nerve of the patient.
Example 9. The system of example 8, wherein the elongated member comprises a nasogastric tube.
Example 10. The system of any of examples 1-9, further comprising one or more sensors, the one or more sensors being configured to sense one or more physiological parameters of a patient, and wherein the processing circuitry is further configured to control the stimulation circuitry based at least in part on the sensed one or more physiological parameters.
Example 11. The system of any of examples 1-10, wherein the system is configured to treat at least one of stroke, traumatic brain injury (TBI), or subarachnoid hemorrhage (SAH).
Example 12. The system of any of examples 1-11, wherein the stimulation circuitry, the memory, and the processing circuitry are housed in a single device.
Example 13. A device comprising: stimulation circuitry configured to generate a first stimulation signal and a second stimulation signal; memory configured to store first stimulation parameters that at least partially define the first stimulation signal and second stimulation parameters that at least partially defined the second stimulation signal; first electrodes configured to transcutaneously or percutaneously deliver the first stimulation signal to a first nerve of a patient; second electrodes configured to transesophageally deliver the second stimulation signal to a second nerve of the patient; and processing circuitry communicatively coupled to the memory, and the stimulation circuitry, the processing circuitry being configured to: control the stimulation circuitry to generate the first stimulation signal; and control the stimulation circuitry to generate the second stimulation signal.
Example 14. The device of example 13, wherein processing circuitry is configured to independently control the stimulation circuitry to generate the first stimulation signal and to generate the second stimulation signal.
Example 15. The device of example 13 or 14, wherein the first stimulation signal is different than the second stimulation signal.
Example 16. The device of any of examples 13-15, wherein the processing circuitry is configured to control the stimulation circuitry to generate the first stimulation signal during a first time period; and control the stimulation circuitry to generate the second stimulation signal during a second time period, wherein the first time period and the second time period are different.
Example 17. The device of any of examples 13-16, wherein the stimulation circuitry is configured to simultaneously generate both the first stimulation signal and the second stimulation signal.
Example 18. The device of any of examples 13-17, further comprising: a patch configured to be applied to skin of the patient, wherein the first plurality of electrodes are disposed on the patch; a nasogastric tube configured to be at least partially inserted into an esophagus of the patient; and an expandable member disposed on the nasogastric tube, wherein the second plurality of electrodes are disposed on the expandable member.
Example 19. The device of any of examples 13-18, wherein the first nerve comprises a trigeminal nerve and the second nerve comprises a vagus nerve.
Example 20. A non-transitory computer readable medium comprising instructions, which when executed, cause processing circuitry to: control stimulation circuitry to generate a first stimulation signal, the first stimulation signal being at least partially defined by first stimulation parameters and being configured to be delivered transcutanously or percutaneously to a first anatomical location; and control the stimulation circuitry to generate a second stimulation signal, the second stimulation signal being at least partially defined by second stimulation parameters and being configured to be delivered transesophageally to a second anatomical location different than the first anatomical location.

Various examples have been described herein. Any combination of the described operations, functions, or features described herein is contemplated. These and other examples are within the scope of the following claims. Based upon the above discussion and illustrations, it is recognized that various modifications and changes may be made to the disclosed examples in a manner that does not require strictly adherence to the examples and applications illustrated and described herein. Such modifications do not depart from the true spirit and scope of various aspects of the disclosure, including aspects set forth in the claims.

1-13. (canceled)

14. A system comprising:
stimulation circuitry configured to generate a first stimulation signal and a second stimulation signal;
memory configured to store first stimulation parameters that at least partially define the first stimulation signal and second stimulation parameters that at least partially defined the second stimulation signal; and
processing circuitry communicatively coupled to the memory, and the stimulation circuitry, the processing circuitry being configured to:
control the stimulation circuitry to generate the first stimulation signal configured to be delivered transcutaneously or percutaneously to a first anatomical location; and
control the stimulation circuitry to generate the second stimulation signal configured to be delivered transesophageally to a second anatomical location different than the first anatomical location.

15. The system of claim 14, wherein processing circuitry is configured to independently control the stimulation circuitry to generate the first stimulation signal and to generate the second stimulation signal.

16. The system of claim 14, wherein the first stimulation signal is different than the second stimulation signal.

17. The system of claim 14, wherein the processing circuitry is configured to control the stimulation circuitry to generate the first stimulation signal during a first time period; and control the stimulation circuitry to generate the second stimulation signal during a second time period, wherein the first time period and the second time period are different.

18. The system of claim 14, wherein the stimulation circuitry is configured to simultaneously generate both the first stimulation signal and the second stimulation signal.

19. The system of claim 14, further comprising a trigeminal neurostimulation (TNS) device, the TNS device comprising at least one electrode, the at least one electrode being configurable for delivery of the first stimulation signal to a trigeminal nerve of a patient.

20. The system of claim 19, wherein the TNS device is configurable to deliver the first stimulation signal to the trigeminal nerve of the patient via at least one of an ophthalmic zone, a maxillary zone, a mandibular zone, or a trigeminal nerve ganglion.

21. The system of claim 14, further comprising:
an elongated member configured to be at least partially inserted into an esophagus of a patient; and
an expandable member having a plurality of electrodes disposed on an outer surface, the plurality of electrodes being configurable for delivery of the second stimulation signal to a vagus nerve of the patient.

22. The system of claim 21, wherein the elongated member comprises a nasogastric tube.

23. The system of claim 14, further comprising one or more sensors, the one or more sensors being configured to sense one or more physiological parameters of a patient, and wherein the processing circuitry is further configured to control the stimulation circuitry based at least in part on the sensed one or more physiological parameters.

24. The system of claim 14, wherein the system is configured to treat at least one of stroke, traumatic brain injury (TBI), or subarachnoid hemorrhage (SAH).

25. The system of claim 14, wherein the stimulation circuitry, the memory, and the processing circuitry are housed in a single device.

26. A device comprising:
stimulation circuitry configured to generate a first stimulation signal and a second stimulation signal;
memory configured to store first stimulation parameters that at least partially define the first stimulation signal and second stimulation parameters that at least partially defined the second stimulation signal;
first electrodes configured to transcutaneously or percutaneously deliver the first stimulation signal to a first nerve of a patient;
second electrodes configured to transesophageally deliver the second stimulation signal to a second nerve of the patient; and
processing circuitry communicatively coupled to the memory, and the stimulation circuitry, the processing circuitry being configured to:
control the stimulation circuitry to generate the first stimulation signal; and
control the stimulation circuitry to generate the second stimulation signal.

27. The device of claim 26, wherein processing circuitry is configured to independently control the stimulation circuitry to generate the first stimulation signal and to generate the second stimulation signal.

28. The device of claim 26, wherein the first stimulation signal is different than the second stimulation signal.

29. The device of claim 26, wherein the processing circuitry is configured to control the stimulation circuitry to generate the first stimulation signal during a first time period; and control the stimulation circuitry to generate the second stimulation signal during a second time period, wherein the first time period and the second time period are different.

30. The device of claim 26, wherein the stimulation circuitry is configured to simultaneously generate both the first stimulation signal and the second stimulation signal.

31. The device of claim 26, further comprising:
a patch configured to be applied to skin of the patient, wherein the first plurality of electrodes are disposed on the patch;
a nasogastric tube configured to be at least partially inserted into an esophagus of the patient; and
an expandable member disposed on the nasogastric tube, wherein the second plurality of electrodes are disposed on the expandable member.

32. The device of claim 26, wherein the first nerve comprises a trigeminal nerve and the second nerve comprises a vagus nerve.

33. A non-transitory computer readable medium comprising instructions, which when executed, cause processing circuitry to:
control stimulation circuitry to generate a first stimulation signal, the first stimulation signal being at least partially defined by first stimulation parameters and being configured to be delivered transcutanously or percutaneously to a first anatomical location; and
control the stimulation circuitry to generate a second stimulation signal, the second stimulation signal being at least partially defined by second stimulation parameters and being configured to be delivered transesophageally to a second anatomical location different than the first anatomical location.
